(19) United States
(12) Patent Application Publication
(10) Pub. No.: US 2023/0405247 A1
(43) Pub. Date: Dec. 21, 2023

(54) ARTIFICIAL RESPIRATION APPARATUS FOR FIRST AID

(71) Applicants: THE FIRST PEOPLE'S HOSPITAL OF WENLING, Zhejiang (CN); TAIZHOU DCTRING NETWORK TECHNOLOGY CO., LTD., Zhejiang Province (CN)

(72) Inventors: Kang LIN, Zhejiang Province (CN); Jie LIN, Zhejiang Province (CN); Xianhua YE, Zhejiang Province (CN); Jingyu HAO, Zhejiang Province (CN); Qianqian JIANG, Zhejiang Province (CN); Xiaoxiao LIN, Zhejiang Province (CN); Huiya QIU, Zhejiang Province (CN); Qiaoqin YAN, Zhejiang Province (CN); Yan LONG, Zhejiang Province (CN); Zisheng LIN, Zhejiang Province (US)

(21) Appl. No.: 18/251,675

(22) PCT Filed: May 31, 2022
(86) PCT No.: PCT/CN2022/096108

§ 371 Date: May 3, 2023

(30) Foreign Application Priority Data
Jun. 1, 2021 (CN) 202110609663.3

(51) Int. Cl. A61M 16/00

(52) U.S. Cl. CPC A61M 16/0048

(57) ABSTRACT

An artificial respiration apparatus for first aid is provided, and relates to the field of medical first-aid supplies. The artificial respiration apparatus comprises an adhesive film, wherein a fixing block is provided on an upper end of the adhesive film; a first adhesive strip is provided on a lower end of the adhesive film; a second adhesive strip is also provided on the lower end of the adhesive film; and a blowing device is inserted in the fixing block. An intraoral ventilation pipe is inserted into the mouth of a person to be rescued, then the adhesive film is applied to the mouth and nose of the person to be rescued, and finally air is blown into a blowing port such that the blown air enters the body of the person to be rescued through a ventilation pipe, and the intraoral ventilation pipe.

### TECHNICAL FIELD

The present disclosure relates to the field of medical first-aid supplies, in particular to an artificial respiration apparatus for first aid.

### BACKGROUND

When someone has a cardiac arrest, the success rate of rescue can be improved by timely cardiac compression and artificial respiration. However, during performing of artificial respiration, a rescuer needs to bend over and kneel down, hold the nose of a person to be rescued with one hand, and completely wrap the mouth of the person to be rescued to blow air. There are several problems during the process.

Firstly, when the rescuer kneels down and bends over, the exertion of blowing is not facilitated, and the condition of the person to be rescued is not easy to observe.

Secondly, due to mouth-to-mouth artificial respiration, the rescuer may suffer from the risk of infection. At the same time, the droplets of the rescuer are easy to blow into the mouth of the person to be rescued.

Thirdly, some rescuers psychologically conflict with contact with the mouth of the person to be rescued, so that timely and effective artificial respiration is not developed smoothly.

At present, there are disposable barrier respiration membranes for artificial respiration in the market, which can isolate direct mouth contact between the rescuer and the person to be rescued and reduce the risk of cross infection. However, there are still some problems. For example, the blown force is poor. The sealing performance is low. The mouth of the rescuer needs to be close to the mouth of the person to be rescued. The mouth of the rescuer completely wraps the mouth of the person to be rescued to blow air. The present disclosure provides an artificial respiration apparatus for first aid so as to solve the problems proposed in the background technology.

### SUMMARY

The purpose of the present disclosure is to provide an artificial respiration apparatus for first aid so as to solve the problems proposed in the background technology.

In order to achieve the above purposes, the present disclosure provides the following technical scheme.

An artificial respiration apparatus for first aid includes an adhesive film, wherein a fixing block is provided on an upper end of the adhesive film, a first adhesive strip is provided on a lower end of the adhesive film, a second adhesive strip is also provided on the lower end of the adhesive film, and a blowing device is inserted in the fixing block; and

the blowing device includes a ventilation pipe, one end of the ventilation pipe passes through the adhesive film and the fixing block in sequence from the lower end of the adhesive film, a first connecting block is provided on the other end of the ventilation pipe, an intraoral ventilation pipe is provided on one end, away from the ventilation pipe, of the first connecting block, a second connecting block is provided on one end, close to the first connecting block, of the intraoral ventilation pipe, the intraoral ventilation pipe is connected with the ventilation pipe through the second connecting block and the first connecting block, a connecting pipe is provided on the upper end of the ventilation pipe and located above the fixing block, an air inlet pipe is provided on one end, away from the ventilation pipe, of the connecting pipe, a filter is provided on one end, away from the connecting pipe, of the air inlet pipe, a blowing port is formed in one end, away from the air inlet pipe, of the filter, a bypass pipe is provided on one side of the ventilation pipe, the upper end of the bypass pipe passes through the adhesive film and the fixing block in sequence, and a protective plug is provided on the upper end of the bypass pipe.

In some embodiments, a plurality of bundling belts are provided on an upper end of the fixing block.

In some embodiments, the second adhesive strip is set as a triangle.

In some embodiments, a blowing cover is provided on one end, away from the filter, of the blowing port.

In some embodiments, a plurality of adhesive tapes are provided on the upper end of the adhesive film, one end of each adhesive tape is fixedly connected with the adhesive film, and the other end of the adhesive tape is adhered with the adhesive film.

In some embodiments, a ventilation port is formed in a surface of the intraoral ventilation pipe, and the ventilation port passes through a shell wall of the intraoral ventilation pipe.

In some embodiments, a double-sided adhesive tape is provided on a lower end of the fixing block, and the fixing block is bonded and connected with the adhesive film through the double-sided adhesive tape.

In some embodiments, an adhesive area is provided on the upper end of the adhesive film and located below the fixing block, and a connecting port is formed inside the adhesive area.

Compared with the prior art, the present disclosure has the following beneficial effects.

Firstly, when the apparatus is used, the intraoral ventilation pipe is inserted into the mouth of a person to be rescued, the intraoral ventilation pipe is bypassed around the tongue of the person to be rescued to prevent the tongue of the person to be rescued from blocking the trachea. Then the adhesive film is applied to the mouth and nose of the person to be rescued. After the adhesive film is adhered to the mouth and nose of the person to be rescued, and through the first adhesive strip and the second adhesive strip, the connecting strength between the adhesive film and the face of the person to be rescued is increased. Finally, air is blown into the blowing port, such that the blown air enters the body of the person to be rescued through the ventilation pipe and the intraoral ventilation pipe, so as to prevent a rescuer from kneeling on the ground or lying prostrate to perform artificial respiration for the person to be rescued, and also prevent the mouth of the rescuer from contacting the mouth of the person to be rescued, thereby making the artificial respiration easier and simpler.

Secondly, after the person to be rescued spontaneously breathes, the adhesive film is torn off from the face of the person to be rescued, then the adhesive film is torn off from the fixing block, the torn adhesive film is discarded, and the blowing device is reserved. By disinfecting the blowing device, the blowing device can be used repeatedly, so that the practicability of the apparatus is improved.

The apparatus has the characteristics of simple structure and convenience in use.

### BRIEF DESCRIPTION OF THE DRAWINGS

FIG. 1 is a structural schematic diagram of an artificial respiration apparatus for first aid.

FIG. 2 is a solid diagram of an artificial respiration apparatus for first aid.

FIG. 3 is a top view of an artificial respiration apparatus for first aid.

FIG. 4 is a structural schematic diagram of an intraoral ventilation pipe in an artificial respiration apparatus for first aid.

FIG. 5 is a structural schematic diagram of a blowing device in an artificial respiration apparatus for first aid.

FIG. 6 is a top view of an adhesive film in an artificial respiration apparatus for first aid.

FIG. 7 is an internal structural schematic diagram of a protective plug in an artificial respiration apparatus for first aid.

Reference signs: 1, adhesive film; 100, adhesive area; 101, connecting port; 2, fixing block; 200, double-sided adhesive tape; 201, bundling belt; 3, first adhesive strip; 4, second adhesive strip; 5, ventilation pipe; 500, first connecting block; 501, intraoral ventilation pipe; 502, second connecting block; 503, ventilation port; 6, bypass pipe; 7, protective plug; 700, pressure relief hole; 701, barrier block; 702, connector; 703, baffle; 704, spring; 8, connecting pipe; 9, air inlet pipe; 10, filter; 11, blowing port; 110, blowing cover; and 12, adhesive tape.

### DETAILED DESCRIPTION OF THE EMBODIMENTS

The following clearly and completely describes the technical scheme in the embodiments of the present disclosure with reference to the attached figures in the embodiments of the present disclosure. Apparently, the described embodiments are merely a part rather than all of the embodiments of the present disclosure. Based on the embodiment in the present disclosure, all other embodiments obtained by the ordinary technical staff in the art under the premise of without contributing creative labor belong to the scope protected by the present disclosure.

### Embodiment 1

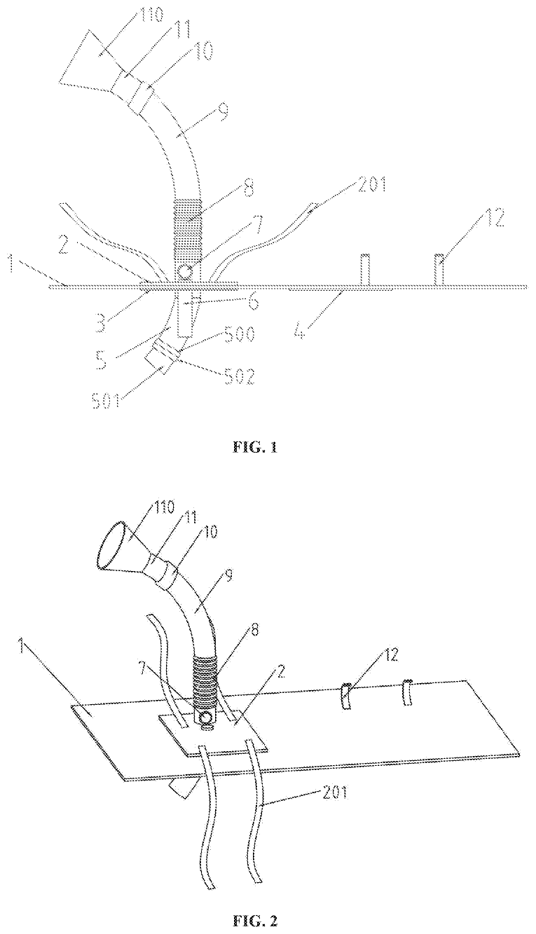

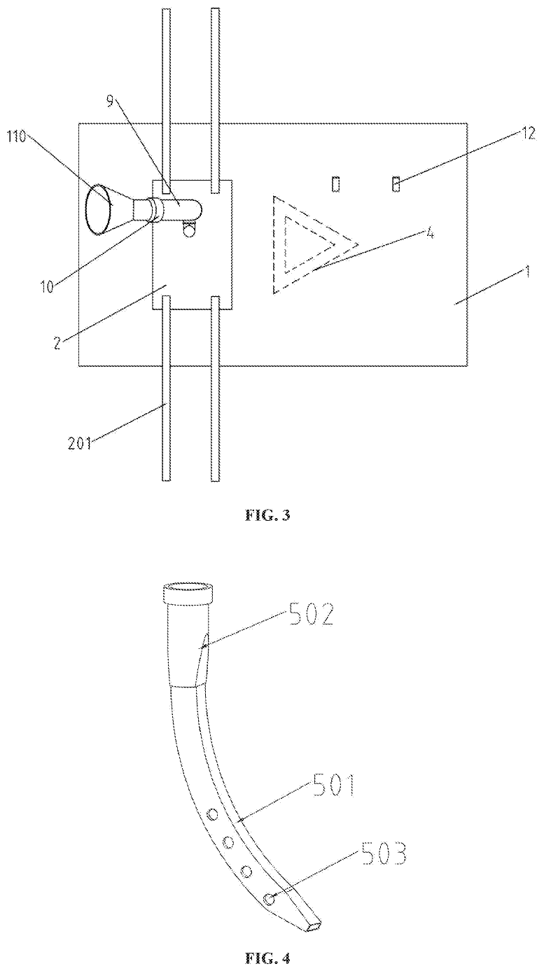

Referring to FIGS. 1 to 3, in the embodiment, an artificial respiration apparatus for first aid includes an adhesive film 1. The lower end of the adhesive film 1 is sticky, and the sticky end of the adhesive film 1 is provided with a protective film. The protective film can prevent foreign matters from sticking to the sticky end of the adhesive film 1. A fixing block 2 is provided on the upper end of the adhesive film 1. The fixing block 2 is made of medical silica gel. A first adhesive strip 3 is provided on the lower end of the adhesive film 1. The first adhesive strip 3 is set as a rectangle. A second adhesive strip 4 is also provided on the lower end of the adhesive film 1. The second adhesive strip 4 is located on the right side of the first adhesive strip 3. A blowing device is inserted in the fixing block 2. The blowing device includes a ventilation pipe 5. One end of the ventilation pipe 5 passes through the adhesive film 1 and the fixing block 2 in sequence from the lower end of the adhesive film 1. A first connecting block 500 is provided on the other end of the ventilation pipe 5. An intraoral ventilation pipe 501 is provided on the end, away from the ventilation pipe 5, of the first connecting block 500. The intraoral ventilation pipe 501 is made of plastic. A second connecting block 502 is provided on the end, close to the first connecting block 500, of the intraoral ventilation pipe 501. The intraoral ventilation pipe 501 and the second connecting block 502 are integrally formed. The intraoral ventilation pipe 501 is connected with the ventilation pipe 5 through the second connecting block 502 and the first connecting block 500. The first connecting block 500 and the second connecting block 502 are in threaded connection. A connecting pipe 8 is provided on the upper end of the ventilation pipe 5 and located above the fixing block 2. The connecting pipe 8 is a plastic pipe. An air inlet pipe 9 is provided on the end, away from the ventilation pipe 5, of the connecting pipe 8. The air inlet pipe 9 is a hose. A filter 10 is provided on the end, away from the connecting pipe 8, of the air inlet pipe 9. A blowing port 11 is formed in the end, away from the air inlet pipe 9, of the filter 10. The blowing port 11 is detachably connected with the filter 10. The blowing port 11 can be connected with a respirator. A blowing cover 110 is provided on the end, away from the filter 10, of the blowing port 11. The blowing cover 10 is made of plastic. The blowing cover 110 is detachably connected with the blowing port 11. A bypass pipe 6 is provided on one side of the ventilation pipe 5. The upper end of the bypass pipe 6 passes through the adhesive film 1 and the fixing block 2 in sequence. The end, passing through the fixing block 2, of the bypass pipe 6 extends to the upper end of the fixing block 2. A protective plug 7 is provided on the end, away from the ventilation pipe 5, of the bypass pipe 6.

In this embodiment, a plurality of bundling belts 201 are provided on the upper end of the fixing block 2. The bundling belt 201 is of elasticity. The bonding strength of the first adhesive strip 3, the second adhesive strip 4 and the adhesive film 1 with the nose and mouth can be enhanced through the bundling belts 201.

In this embodiment, the second adhesive strip 4 is set as a triangle. The second adhesive strip 4 is set as a triangle to facilitate the second adhesive strip 4 to be attached to the nose of a person to be rescued.

In this embodiment, a plurality of adhesive tapes 12 are provided on the upper end of the adhesive film 1. One end of the adhesive tape 12 is fixedly connected with the adhesive film 1, and the other end of the adhesive tape 12 is adhered with the adhesive film 1. The connecting pipe 8 is bent so that the air inlet pipe 9 is bonded to the surface of the adhesive film 1, and then the air inlet pipe 9 is fixed to the surface of the adhesive film 1 through the adhesive tape 12, thereby reducing the occupied space of the apparatus. The apparatus is easier to carry.

### Embodiment II

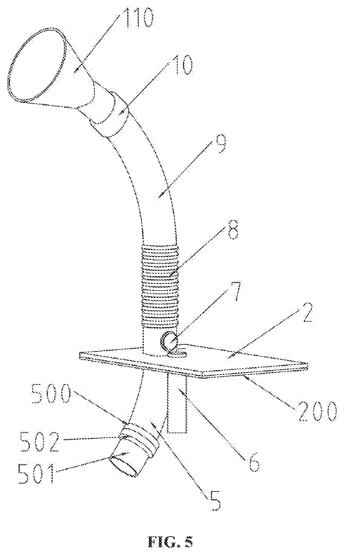

Referring to FIG. 4, the difference between the second embodiment and the first embodiment lies in that a ventilation port 503 is formed in the surface of the intraoral ventilation pipe 501, and the ventilation port 503 passes through the shell wall of the intraoral ventilation pipe 501. The number of the ventilation ports 503 is two.

### Embodiment III

Referring to FIG. 5 and FIG. 6, the difference between the third embodiment and the first embodiment lies in that a double-sided adhesive tape 200 is provided on the lower end of the fixing block 2. The double-sided adhesive tape 200 is a disposable product and can be replaced at any time. The fixing block 2 is bonded and connected with the adhesive film 1 through the double-sided adhesive tape 200. An adhesive area 100 is provided on the upper end of the adhesive film 1 and located below the fixing block 2, and a connecting port 101 is formed inside the adhesive area 100. The connecting port 101 passes through the adhesive film 1.

### Embodiment IV

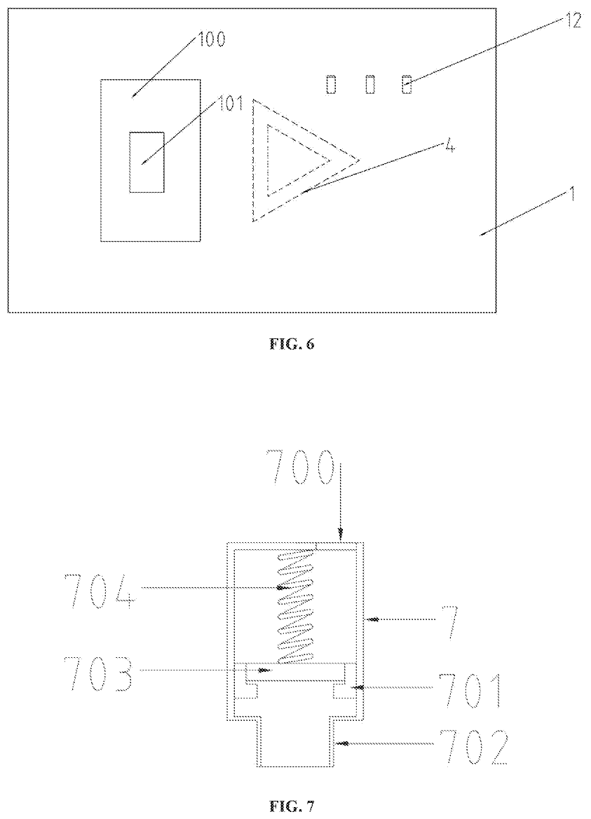

Referring to FIG. 7, the difference between the fourth embodiment and the first embodiment lies in that a pressure limiting structure is provided inside the protective plug 7. The pressure limiting structure includes a pressure relief hole 700. The pressure relief hole 700 is located at the upper end of the protective plug 7. A connector 702 is provided on the lower end of the protective plug 7. A barrier block 701 is provided inside the protective plug 7. A circular hole is formed in the middle part of the barrier block 701. A baffle 703 is movably connected into the circular hole. A spring 704 is provided on the upper end of the baffle 703. One end of the spring 704 is connected with the inner top end of the protective plug 7, and the other end of the spring 704 is connected with the baffle 703.

The working process of the present disclosure is as follows.

When the apparatus is used, the intraoral ventilation pipe 501 is firstly inserted into the mouth of the person to be rescued. The intraoral ventilation pipe 501 is bypassed around the tongue of the person to be rescued to prevent the tongue of the person to be rescued from blocking the trachea. Then, the adhesive film 1 is applied to the mouth and nose of the person to be rescued. After the adhesive film 1 is adhered to the mouth and nose of the person to be rescued, and through the first adhesive strip and the second adhesive strip, the connecting strength between the adhesive film 1 and the face of the person to be rescued is increased. The bundling belts 201 are bypassed around the neck to be fixed, so that the bonding strength between the adhesive film 1 and the face of the person to be rescued is further increased. Finally, air is blown into the blowing port 11. The blown air enters the body of the person to be rescued through the ventilation pipe 5 and the intraoral ventilation pipe 501. The ventilation port 503 in the intraoral ventilation pipe 501 can prevent the tongue from blocking the air inlet of the intraoral ventilation pipe 501. When it is necessary to pump out liquid accumulated in the oral cavity of the person to be rescued, a pipette can be inserted into the oral cavity through the bypass pipe 6, and then the liquid in the oral cavity of the person to be rescued can be sucked out by using the pipette.

After the person to be rescued spontaneously breathes, the adhesive film 1 is torn off from the face of the person to be rescued, then the adhesive film 1 is torn off from the fixing block 2, the torn adhesive film 1 is discarded, and the blowing device is reserved. By disinfecting the blowing device, the blowing device can be used repeatedly.

The above mentioned are only preferred specific embodiments of the present disclosure. However, the scope of protection of the present disclosure is not limited to the embodiments described herein. Any technicians skilled in the technical field are within the technical scope disclosed by the present disclosure; and any replacements or modifications according to the technical schemes of the present disclosure and ideas thereof all shall be included in the scope of protection of the present disclosure.

1. An artificial respiration apparatus for first aid, comprising an adhesive film, wherein a fixing block is provided on an upper end of the adhesive film (1), a first adhesive strip is provided on a lower end of the adhesive film, a second adhesive strip is also provided on the lower end of the adhesive film and a blowing device is inserted in the fixing block;
the blowing device comprises a ventilation pipe one end of the ventilation pipe passes through the adhesive film and the fixing block, in sequence from the lower end of the adhesive film a first connecting block is provided on an other end of the ventilation pipe, an intraoral ventilation pipe is provided on one end, away from the ventilation pipe, of the first connecting block, a second connecting block is provided on one end, close to the first connecting block, of the intraoral ventilation pipe the intraoral ventilation pipe is connected with the ventilation pipe through the second connecting block; and the first connecting block, a connecting pipe is provided on an upper end of the ventilation pipe and located above the fixing block, an air inlet pipe is provided on one end, away from the ventilation pipe; of the connecting pipe a filter is provided on one end, away from the connecting pipe, of the air inlet pipe, a blowing port is formed in one end, away from the air inlet pipe of the filter, a bypass pipe is provided on one side of the ventilation pipe, an upper end of the bypass pipe passes through the adhesive film and the fixing block in sequence, and a protective plug is provided on the upper end of the bypass pipe.

2. The artificial respiration apparatus for first aid according to claim 1, wherein a plurality of bundling belts are provided on an upper end of the fixing block.

3. The artificial respiration apparatus for first aid according to claim 1, wherein the second adhesive strip is set as a triangle.

4. The artificial respiration apparatus for first aid according to claim 1, wherein a blowing cover is provided on one end, away from the filter of the blowing port.

5. The artificial respiration apparatus for first aid according to claim 1, wherein a plurality of adhesive tapes are provided on the upper end of the adhesive film one end of each of the adhesive tapes is fixedly connected with the adhesive film, and an other end of each of the adhesive tapes is adhered to the adhesive film.

6. The artificial respiration apparatus for first aid according to claim 1, wherein a ventilation port is formed in a surface of the intraoral ventilation pipe, and the ventilation port passes through a shell wall of the intraoral ventilation pipe.

7. The artificial respiration apparatus for first aid according to claim 1, wherein a double-sided adhesive tape is provided on a lower end of the fixing block, and the fixing block is bonded and connected to the adhesive film through the double-sided adhesive tape.

8. The artificial respiration apparatus for first aid according to claim 1, wherein an adhesive area is provided on the upper end of the adhesive film and located below the fixing block, and a connecting port is formed inside the adhesive area.
